SEQUENCE LISTING (27 sequences)

SEQ ID NO: 1 - DNA, 20 bp, Artificial Sequence
  Synthetic
tggcggtcacaattaggtca

SEQ ID NO: 2 - DNA, 24 bp, Artificial Sequence
  Synthetic
ttgtgaccgccatttctcgtttct

SEQ ID NO: 3 - DNA, 22 bp, Artificial Sequence
  Synthetic
agttttcagaaataagatggtt

SEQ ID NO: 4 - DNA, 22 bp, Artificial Sequence
  Synthetic
tctgaaaacttctgacctaatt

SEQ ID NO: 5 - DNA, 24 bp, Artificial Sequence
  Synthetic
aacggctgcactgactcacatgat

SEQ ID NO: 6 - DNA, 26 bp, Artificial Sequence
  Synthetic
tcagtgcagccgttgcatcgtcacca

SEQ ID NO: 7 - DNA, 32 bp, Artificial Sequence
  Synthetic
cacacagctcttcggccagcaaaagcagggta

SEQ ID NO: 8 - DNA, 38 bp, Artificial Sequence
  Synthetic
cacacagctcttctattagtagaaacaagggtattttt

SEQ ID NO: 9 - DNA, 32 bp, Artificial Sequence
  Synthetic
cacacagctcttctattagcaaaagcagggtg

SEQ ID NO: 10 - DNA, 37 bp, Artificial Sequence
  Synthetic
cacacagctcttcggccagtagaaacaagggtgtttt

SEQ ID NO: 11 - DNA, 32 bp, Artificial Sequence
  Synthetic
cacacagctcttctattagcaaaagcaggagt

SEQ ID NO: 12 - DNA, 38 bp, Artificial Sequence
  Synthetic
cacacagctcttcggccagtagaaacaaggagtttttt

SEQ ID NO: 13 - DNA, 31 bp, Artificial Sequence
  Synthetic
cacacagctcttctattagcaaaagcagggg

SEQ ID NO: 14 - DNA, 37 bp, Artificial Sequence; identical to SEQ ID NO: 10
  Synthetic

SEQ ID NO: 15 - DNA, 21 bp, Artificial Sequence
  Synthetic
tagtcctcttctctctccttg

SEQ ID NO: 16 - DNA, 21 bp, Artificial Sequence
  Synthetic
caaggagagagaagaggacta

SEQ ID NO: 17 - DNA, 22 bp, Artificial Sequence
  Synthetic
cgaaatcccaggcctatttggt

SEQ ID NO: 18 - DNA, 22 bp, Artificial Sequence
  Synthetic
accaaataggcctgggatttcg

SEQ ID NO: 19 - DNA, 12 bp, Artificial Sequence
  Synthetic
agcaaaagcagg

SEQ ID NO: 20 - DNA, 366 bp, Artificial Sequence
  Synthetic
atggatccaaacactgtgtcaagctttcaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcggtcacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttag

SEQ ID NO: 21 - DNA, 366 bp, Artificial Sequence
  Synthetic
atggatccaaacactgtgtcaagctttcaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagttttcagaaataagatggttgattgaagaagtgagacacaaactgaagataacagagaatagttttgagcaaataacatttatgcaagccttacatctattgcttgaagtggagcaagagataagaactttctcgtttcagcttatttag

SEQ ID NO: 22 - DNA, 366 bp, Artificial Sequence
  Synthetic
atggatccaaacactgtgtcaagctttcaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcggtcacaattaggtcagaagttttcagaaataagatggttgattgaagaagtgagacacaaactgaagataacagagaatagttttgagcaaataacatttatgcaagccttacatctattgcttgaagtggagcaagagataagaactttctcgtttcagcttatttag

SEQ ID NO: 23 - DNA, 1497 bp, Artificial Sequence
  Synthetic
atggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctgcactgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaa

SEQ ID NO: 24 - protein, 121 aa, Artificial Sequence
  Synthetic
MDPNTVSSFQDILLRMSKMQLESSSEDLNGMITQFESLKLYRDSLGEAVMRMGDLHSLQNRNEKWRSQLGQKFEEIRWLIEEVRHKLKITENSFEQITFMQALHLLLEVEQEIRTFSFQLI

SEQ ID NO: 25 - protein, 121 aa, Artificial Sequence
  Synthetic
MDPNTVSSFQDILLRMSKMQLESSSEDLNGMITQFESLKLYRDSLGEAVMRMGDLHSLQNRNEKWREQLGQKFSEIRWLIEEVRHKLKITENSFEQITFMQALHLLLEVEQEIRTFSFQLI

SEQ ID NO: 26 - protein, 121 aa, Artificial Sequence
  Synthetic
MDPNTVSSFQDILLRMSKMQLESSSEDLNGMITQFESLKLYRDSLGEAVMRMGDLHSLQNRNEKWRSQLGQKFSEIRWLIEEVRHKLKITENSFEQITFMQALHLLLEVEQEIRTFSFQLI

SEQ ID NO: 27 - protein, 498 aa, Artificial Sequence
  Synthetic
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAALTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

(19) United States
(12) Patent Application Publication
(10) Pub. No.: US 2014/0227310 A1
(43) Pub. Date: Aug. 14, 2014

(54) RECOMBINANT INFLUENZA VIRUS HIGHLY EXPRESSING HA PROTEIN AND PREPARATION METHOD AND USE THEREOF

(71) Applicants: Zejun Li, Shanghai (CN); Qiaoyang Teng, Shanghai (CN)

(72) Inventors: Zejun Li, Shanghai (CN); Qiaoyang Teng, Shanghai (CN)

(73) Assignee: SHANGHAI VETERINARY RESEARCH INSTITUTE, CAAS, Shanghai (CN)

(21) Appl. No.: 14/343,605

(22) PCT Filed: Sep. 4, 2012
(86) PCT No.: PCT/CN2012/080965

§ 371 Date: Mar. 7, 2014

(30) Foreign Application Priority Data
Sep. 8, 2011 (CN) 201110266082.0

(51) Int. Cl. C12N 7/00

(52) U.S. Cl. CPC C12N 7/00; C12N 2760/16021
USPC 424/186.1; 435/235.1

(57) ABSTRACT

A PR8 recombinant influenza virus contains an HA and/or NA gene of H1, H3, H4, H5, H6, H7, H9 or H10 subtype influenza virus, and 6 internal genes (PB1, PB2, PA, NP, M and NS genes) of PR8 virus, in which the NS and/or NP gene have the following mutation sites: the NS2 protein encoded by the NS gene has an E67S point mutation, E74S point mutation, or E67S/E74S point mutation, and the NP protein encoded by the NP gene has a G132A point mutation.

### FIELD OF THE INVENTION

The present invention belongs to the field of biotechnology, and relates to the vaccine production field, in particular to a recombinant influenza virus highly expressing HA protein, and a preparation method and use thereof.

### BACKGROUND OF THE INVENTION

Influenza is an acute and highly contagious disease caused by influenza A virus in the family of orthomyxoviridae influenza viruses, and highly pathogenic avian influenza has been identified as being included in the List A of OIE (International Office of Epizootics). Influenza viruses can be classified into three types: A, B and C based on different matrix proteins (M). Influenza viruses can also be classified into different subtypes based on their differences in hemagglutinin (HA) and neuraminidase (NA) antigenicities, and the influenza A virus is classified into 16 subtypes based on HA and into 9 subtypes based on NA. Being highly contagious and droplet-transmissible, influenza viruses could outbreak suddenly within a short period of time and then spread very fast, causing disease epidemics of different magnitudes, and even a worldwide epidemic. The outbreaks of highly pathogenic avian influenza in Pennsylvania during 1983-1984 led to 17 million poultry deaths and loss of nearly 65 million dollars. By the end of 2003, there were outbreaks of highly pathogenic avian influenza in many Asian countries and regions. These outbreaks of avian influenza made more than 100 million poultry dead or get culled, and a sharp decline in poultry meat production and sales volume emerged in some countries, causing price falling and suspension of the import and export of poultry meat and products thereof; in addition, the poultry farming industry, feed industry and tourism were also affected adversely. It was estimated by FAO (Food and Agriculture Organization) that the avian influenza-resulting loss was at least 500 million dollars. Furthermore, plenty of subtype avian influenza viruses have the ability of cross-host transmission to human, therefore, the outbreaks of this disease also endangered social public safety at the same time. Three influenza pandemics during the 20th century are the Spanish flu (H1N1) in 1918, the Asian flu (H2N2) in 1957 and the Hong Kong flu (H3N2) in 1968, of which the largest is the Spanish flu. This influenza pandemic caused 20 million deaths worldwide, which is more than the death toll of the World War I, and it also ranks first among all the world's infectious diseases in the mortality aspect.

The genome of influenza virus consists of single-stranded, negative-sense RNA fragments. Influenza A virus is divided into eight fragments, which encode 11 functional proteins; three polymerase proteins PB2, PB1 and PA are encoded by the fragments 1, 2 and 3 respectively; hemagglutinin HA is encoded by the fragment 4; nucleocapsid protein NP is encoded by the fragment 5; neuraminidase NA is encoded by the fragment 6; matrix protein M1 and ion channel M2 are encoded by the fragment 7; nonstructural proteins NS1 and NS2 are encoded by the fragment 8. Among them, the HA and NA proteins are two major surface glycoproteins in influenza virus, and the HA protein is the most important protective antigen in influenza virus.

Currently, different measures have been taken for prevention and control of animal and human influenzas in different countries, but vaccination is still the best choice for influenza prevention, hence, development of an effective influenza vaccine brings an extremely important significance for controlling influenza epidemics. Inactivated whole virus vaccines are the vaccines that are used most widely at present, and such vaccines are high in safety, free from the risks of reversion of virulence and mutation, and capable of surviving the assaults from the same subtype influenza virus. At present, those influenza vaccines that have been available on the market are essentially prepared by being cultured in chicken embryos, and this approach of culturing vaccine in chicken embryos has already existed domestically and overseas for 50 years. This chicken embryo-type influenza vaccine production approach requires a large amount of chicken embryos that probably result in potential contamination, moreover, it has an unacceptable long culture period, hardly expands the yield and is in a disadvantageous position in tackling an outbreak of influenza on a large scale. As a result of this, the World Health Organization, the U.S. government and other institutions all encouraged the development of cell-culture technologies to produce influenza vaccines by taking the place of the current chicken embryo culture technology. To accelerate the development of cell-culture influenza vaccine technologies, the U.S. government has decided to invest 1.1 billion dollars to fund six major companies to develop new influenza vaccine technologies, including GlaxoSmithKline, edimmune, Novartis, DynPort, Solvay and Pasteur. In 2007, one of the world's largest biopharmaceutical companies Novartis announced that its influenza vaccine for human use, i.e. Optaflu, was commercially available and was the only one approved (EU-approved) cell-culture influenza vaccine for human use, making it one of the most significant innovations in the 50-year influenza vaccine production history.

No matter whether the virus is prepared using the chicken embryo method or the large-scale cell preparation method, the critical factor is whether the vaccine virus seed itself is a high-yield virus strain or not. A/Puerto Rico/8/34 (PR8) is a chicken embryo-culture virus strain, one of the high-yield strains on chicken embryos at present; and 6 internal genes of the PR8 and HA and NA genes of the pandemic virus strain are often recombined (6+2 mode) in vaccine development, and the recombinant virus is used as a vaccine strain to improve viral titer. In order to further improve the virus titer of the PR8 to meet the great demand for vaccines during an influenza outbreak, researchers have conducted extensive researches to improve the yield of the virus strain through optimization for virus genes. It is found by these researches that certain amino acid sites of some proteins of this influenza virus place an important effect upon the multiplication capacity of this virus, e.g. tyrosine (Tyr) at the 360^{th} site on PB2 and glutamic acid (Glu) at the 55^{th} site on NS1 also play a role.

### SUMMARY OF THE INVENTION

An object of the present invention is to provide a PR8 mutant recombinant influenza virus, which is capable of highly expressing HA protein and suitable for large-scale production of influenza vaccine.

To reach the abovementioned object, adopted is the technical solution below:

A PR8 recombinant influenza virus, comprising an HA and/or NA gene of H1 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation, and the H1 subtype influenza virus is H1 subtype influenza virus other than PR8 virus (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H3 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H4 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H5 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E678 point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H6 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H7 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H9 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

A PR8 recombinant influenza virus, comprising an HA and/or NA gene of H10 subtype influenza virus, and 6 internal genes (PB1, PB2, PA, NP, M and NS genes) of PR8 virus, wherein the NS and/or NP gene comprises the following mutation sites: the NS2 protein encoded by the NS gene has an E67S point mutation, E74S point mutation or E67S/E74S point mutation, and the NP protein encoded by the NP gene has a G132A point mutation (the NS gene that encodes the NS2 protein having the E67S point mutation, E74S point mutation or E67S/E74S point mutation as well as the NP gene that encodes the NP protein having the G132A point mutation have nucleotide sequences as shown in SEQ ID. NO:20-23 respectively, and amino acid sequences as shown in SEQ ID. NO:24-27, respectively).

Another object of the present invention is to provide a preparation method of the PR8 recombinant influenza virus.

To reach the abovementioned object, adopted is the technical solution below:

A preparation method of the PR8 recombinant influenza virus, comprising:

constructing recombinant plasmids comprising the HA and NA genes of H1, H3, H4, H5, H6, H7, H9 and H10 subtype influenza viruses respectively;

constructing a recombinant plasmid comprising a PR8 virus mutant gene fragment, the PR8 virus mutant gene fragment being selected from the group consisting of the following mutant NS or NP gene fragments: a PR8 virus NS gene fragment encoding the NS2 protein containing the E67S point mutation, E74S point mutation or E67S/E74S point mutation, and a PR8 virus NP gene fragment encoding the NP protein containing the G132A point mutation;

co-transfecting the recombinant plasmids of the HA and NA genes of the various subtype influenza viruses above, the recombinant plasmid comprising the PR8 virus mutant gene fragment, and the plasmids respectively comprising the PA, PB1, PB2, M, NP or NS internal gene of PR8 virus, into a 293T cell, and culturing the transfected cell;

inoculating the cultured cell supernatant into chicken embryos, and culturing the chicken embryos in an incubator for a proper time period to acquire chicken embryo allantoic fluid, detecting the hemagglutination condition of the allantoic fluid, and in case of the presence of hemagglutinin activity, determining the absence of unexpected mutations by sequence analysis to acquire the PR8 recombinant influenza virus.

In a specific embodiment of the present invention, a preparation method of the PR8 recombinant influenza virus comprises:

(1) Constructing recombinant plasmids:

A. HA and NA genes of H1, H3, H4, H5, H6, H7, H9, H10 subtype influenza viruses are acquired;

The method for acquiring the HA and NA genes of the H1, H3, H4, H5, H6, H7, H9, H10 subtype influenza viruses is as follows:

Total RNA of the H1, H3, H4, H5, H6, H9 and H10 subtype influenza viruses is extracted respectively;

By taking the total RNA as the template, cDNA of the H1, H3, H4, H5, H6, H9 and H10 subtype influenza viruses is synthesized through reverse transcription respectively;

By taking the acquired cDNA as the template, SEQ ID. NO:13 and SEQ ID. NO:14 as well as SEQ ID. NO:11 and SEQ ID. NO:12 are used as upstream and downstream primers respectively for amplification, to acquire the HA and NA genes of the H1, H3, H4, H5, H6, H9 and H10 subtype influenza viruses;

By taking the cDNA of the H5 subtype influenza virus as the template, SEQ ID. NO:15 and SEQ ID. NO:13 primers as well as SEQ ID. NO:16 and SEQ ID. NO:14 primers at mutant H5HA alkaline cleavage sites are used respectively for amplification, and then, SEQ ID. NO:13 and SEQ ID. NO:14 primers are used for PCR fusion amplification to acquire the HA gene of the H5N1 subtype influenza virus containing low pathogenic avian influenza strain alkaline cleavage sites;

The preparation of the HA and NA genes of the H7 subtype influenza virus is as follows: the NA gene and the HA gene (which contains low pathogenic avian influenza strain alkaline cleavage sites) of the H7 subtype influenza virus are artificially synthesized, and SEQ ID. NO:13 and SEQ ID. NO:14 as well as SEQ ID. NO:11 and SEQ ID. NO:12 are used as upstream and downstream primers for amplification respectively, to acquire the HA and NA genes of the H7 subtype influenza virus.

B. A PR8 virus NP gene fragment encoding an NP protein having a G132A point mutation and a PR8 virus NS gene fragment encoding an NS2 protein having an E67S, E74S or NS2E67/74S point mutation are acquired respectively;

Preferably, the preparation for acquiring the PR8 virus NP gene fragment encoding the NP protein having the G132A point mutation and the PR8 virus NS gene fragment encoding the NS2 protein having the E67S, E74S or NS2E67/74S point mutation is as follows:

By taking the recombinant plasmid of a PR8 virus NP gene as the template, primers SEQ ID. NO:7 and SEQ ID. NO:6 as well as primers SEQ ID. NO:5 and SEQ ID. NO:8 are used respectively for PCR amplification under the action of Pfx DNA polymerase; by taking two segments of the PCR product as the template, SEQ ID. NO:7 and SEQ ID. NO:8 are used as primers for PCR amplification for the second time, to acquire the PR8 virus NP gene fragment having the G132A point mutation;

By taking the PBD-PR8NS recombinant plasmid as the template, primers SEQ ID. NO:9 and SEQ ID. NO:2 as well as primers SEQ ID. NO:1 and SEQ ID. NO:10 are used respectively for PCR amplification under the action of Pfx DNA polymerase; by taking two segments of the PCR product as the template, SEQ ID. NO:9 and SEQ ID. NO:10 are used as the primers for PCR fusion for the second time, to acquire the NS gene fragment encoding the NS2 protein having the E67S site-directed mutation;

By taking the PBD-PR8NS recombinant plasmid as the template, primers SEQ ID. NO:9 and SEQ ID. NO:4 as well as primers SEQ ID. NO:3 and SEQ ID. NO:10 are used respectively for PCR amplification under the action of Pfx DNA polymerase; by taking two segments of the PCR product as the template, SEQ ID. NO:9 and SEQ ID. NO:10 are used as the primers for PCR fusion for the second time, to acquire the NS gene fragment encoding the NS2 protein having the E74S site-directed mutation;

By taking the NS gene fragment encoding the NS2 protein having the E67S site-directed mutation as the template, primers SEQ ID. NO:9 and SEQ ID. NO:4 as well as primers SEQ ID. NO:3 and SEQ ID. NO:10 are used respectively for PCR amplification under the action of Pfx DNA polymerase; by taking two segments of the PCR product as the template, SEQ ID. NO:9 and SEQ ID. NO:10 are used as the primers for PCR fusion for the second time, to acquire the NS gene fragment encoding the NS2 protein simultaneously having the E67S site-directed mutation and the E74S site-directed mutation;

C. The recombinant plasmids are prepared: the PR8 virus NP gene fragment encoding the NP protein having the G132A point mutation, the PR8 virus NS gene fragment encoding the NS2 protein having the E67S, E74S or NS2E67174S point mutation, and the acquired HA and NA genes above are subject to enzyme digestion, ligation and transformation to acquire corresponding recombinant plasmids; the recombinant plasmids are more than one of the group consisting of: PBD-(H1)HA; PBD-(H1)NA; PBD-(H3)HA, PBD-(H3)NA; PBD-(H4)HA, PBD-(H4N2)NA; PBD-(H5)HA, PBD-(H5)NA; PBD-(H6)HA, PBD-(H6)NA; PBD-(H7)HA, PBD-(H7)NA; PBD-(H9)HA, PBD-(H9)NA; PBD-(H10)HA, PBD-(H10)NA; PBD-PR8NS-NS2E67/74S, PBD-PR8NS-NS2E67S, PBD-PR8NS-NS2E74S, PBD-PR8NP-G132A, PBD-PR8PB1, PBD-PR8PB2, PBD-PR8PA, PBD-PR8NP, PBD-PREM and PBD PR8NS (Zejun Li, et al. JVI, 2005, 79(18): 12058-12064).

(2) Preparing the PR8 recombinant influenza virus: the acquired recombinant plasmids above are transfected into a 293T cell in accordance with corresponding combinations; the transfected cell supernatant is treated with TPCK-Trypsin and then inoculated into SPF chicken embryos, followed by culture; and chicken embryo allantoic fluid is acquired to acquire the abovementioned PR8 recombinant influenza virus.

Another object of the present invention is to provide use of the abovementioned PR8 recombinant influenza virus.

The technical solution is specifically as follows:

Use of the abovementioned PR8 recombinant influenza virus in preparation of influenza vaccine.

It is found by the inventor of the present inventions that, the multiplication capacity of the virus mutant strain on chicken embryos is improved significantly when amino acid at the 67^{th} site of the NS2 protein of the PR8 virus strain is mutated from E to S (E67S point mutation), or amino acid at the 74^{th} site of the NS2 protein is mutated from E to S (E74S point mutation), or amino acids at the 64^{th} and 74^{th} sites of the NS2 protein are simultaneously mutated from E to S (E67S/E74S point mutation). In addition, the multiplication capacity of the virus mutant strain on cells is improved significantly when amino acid at the 132^{nd} site of the NP protein is mutated from G to A (G132A point mutation). The recombinant virus highly expressing HA antigen in the present invention is acquired by artificial recombination of the internal genes of these mutation viruses with high multiplication capacity and the different subtype (H1, H3, H4, H5, H6, H7, H9, H10) influenza viruses, and these recombinant viruses can be used in large-scale preparation of influenza vaccine.

### BRIEF DESCRIPTION OF THE DRAWINGS

The present invention will be further illustrated below in details in conjunction with the accompanying drawings and embodiments.

FIG. 1 illustrates the hemagglutinin activities of the recombinant PR8 mutation viruses and the recombinant PR8 viruses on chicken embryos.

FIG. 2 illustrates the hemagglutinin activities of the recombinant PR8 mutation viruses and the recombinant PR8 viruses on cells.

### DETAILED DESCRIPTION OF THE INVENTION

### Example 1

### Construction and Identification of Recombinant Plasmids

1. Primer Design

Mutant primers of NS and NP of the influenza virus PR8 are designed; a 12 bp reverse transcription primer of influenza virus, universal primers of influenza A, and mutant primers at H5 and H7HA cleavage sites are designed by our lab. Shown in Table 1 are the specific sequences of the abovementioned primers (for primer sequences used in the present invention, see Table 1), which are all synthesized by Shanghai Invitrogen.

2. Site-Directed Mutations at Two Sites

Nucleotides at expected mutation amino acid sites are mutated using a two-step PCR process. At first, by taking PBD-PR8NP as the template, BSPQI-NP-forward and PR8-NP-400R as well as PR8-NP-387F and BSPQI-NP-reverse are used as upstream and downstream primers respectively for PCR amplifications under the action of Pfx DNA polymerase (Invitrogen). Two fragments resulted from PCR are recovered using a gel extraction kit. By taking the two recovered fragments of the PCR product as the template, BSPQI-NP-forward and BSPQI-NP-reverse are used as primers for PCR fusion for the second time. In this way, an NP gene fragment encoding an NP protein having a G132A point mutation is acquired. The PCR amplification protocol is as follows: pre-degenerate at 94° C. for 5 minutes, enter the following cycle: degenerate at 94° C. for 45 seconds, anneal at 53° C. for 45 seconds and extend at 72□ for 1 minute to 1 minute and 45 seconds, complete this cycle 30 times, and finally, extend at 72° C. for 10 minutes. At the end of reaction, the PCR product undergoes electrophoresis on 1.0% agarose gel.

In the same way, NS2 mutant primers in Table 1: PR8-NS2-193F and PR8-NS2-204R as well as PR8-NS2-215F and PR8-NS2-224R, and NS gene amplification primers: BSPQI-NS-forward and BSPQI-NS-reverse are used to acquire NS gene PCR amplification products having NS2E67S, E74S and NS2E67174S point mutations, respectively.

Specifically, by taking the PBD-PR8NS recombinant plasmid as the template, primers SEQ ID. NO:9 and SEQ ID. NO:2 as well as primers SEQ ID. NO:1 and SEQ ID. NO:10 are used respectively for PCR amplification under the action of Pfx DNA polymerase; by taking two segments of the PCR product as the template, SEQ ID. NO:9 and SEQ ID. NO:10 are used as the primers for PCR fusion for the second time, to acquire the NS gene fragment encoding the NS2 protein having the E67S site-directed mutation;

By taking the PBD-PRBNS recombinant plasmid as the template, primers SEQ ID. NO:9 and SEQ ID. NO:4 as well as primers SEQ ID. NO:3 and SEQ ID. NO:10 are used respectively for PCR amplification under the action of Pfx DNA polymerase; by taking two segments of the PCR product as the template, SEQ ID. NO:9 and SEQ ID, NO:10 are used as the primers for PCR fusion for the second time, to acquire the NS gene fragment encoding the NS2 protein having the E74S site-directed mutation;

By taking the NS gene fragment encoding the NS2 protein having the E67S site-directed mutation as the template, primers SEQ ID. NO:9 and SEQ ID. NO:4 as well as primers SEQ ID, NO:3 and SEQ ID. NO:10 are used respectively for PCR amplification under the action of Pfx DNA polymerase; by taking two segments of the PCR product as the template, SEQ ID. NO:9 and SEQ ID. NO:10 are used as the primers for PCR fusion for the second time, to acquire the NS gene fragment encoding the NS2 protein simultaneously having the E67S site-directed mutation and the E74S site-directed mutation;

3. PCR Amplifications of the HA and NA Genes

The HA and NA genes originate from different subtype influenza viruses (HxNy, representing H1N1, H3N2, H4N2, H5N1, H6N4, H7N7, H9N2 and H10N8 subtype influenza viruses). Total RNA of all the viruses is extracted using Trizol (Invitrogen) except for the H7N7 subtype influenza.

With a 12-bp primer 5′-AGCAAAAGCAGG-3′ (Table 1) serving as the specific primer, first-strand cDNA is synthesized using a reverse transcription system kit (TakaRa) according to its instruction. By taking the acquired first-strand cDNA as the template, BSPQI-HA-forward and BSPQI-HA-reverse as well as BSPQI-NA-forward and BSPQI-NA-reverse are used as upstream and downstream primers (containing BspQI enzyme digestion sites, as shown in Table 1) for amplifications, to acquire the HA genes of the H1N1, H3N2, H4N2, H6N4, H9N2 and H10N8 subtype influenza viruses and the NA genes of the H1N1, H3N2, H4N2, H5N1, H6N4, H9N2 and H10N8 subtype influenza viruses of the fragments, respectively. The PCR amplification protocol is as follows: pre-degenerate at 94° C. for 5 minutes, enter the following cycle: degenerate at 94° C. for 45 seconds, anneal at 53° C. for 45 seconds and extend at 72° C. for 1 minute and 45 seconds, complete this cycle 30 times, and finally, extend at 72° C. for 10 minutes. At the end of reaction, the PCR product undergoes electrophoresis on 1.0% agarose gel.

After the HA gene of the H5N1 subtype influenza virus is acquired through extraction, the downstream primers H5-reverse and BSPQI-HA-forward at mutant H5HA alkaline cleavage sites as well as the upstream primers H5-forward and BSPQI-HA-reverse at mutant alkaline cleavage sites are used respectively for PCR amplifications, and then, the primers BSPQI-HA-forward and BSPQI-HA-reverse are used for PCR fusion. The PCR amplification protocol is as follows: pre-degenerate at 94° C. for 5 minutes, enter the following cycle: degenerate at 94° C. for 45 seconds, anneal at 53° C. for 45 seconds and extend at 72° C. for 1 minute and 45 seconds, complete this cycle 30 times, and finally, extend at 72° C. for 10 minutes. At the end of reaction, the PCR product undergoes electrophoresis on 1.0% agarose gel. The HA gene of H5 containing alkaline cleavage sites of the low pathogenic avian influenza virus strain is acquired.

In this study, the NA gene of the H7N7 influenza virus and the HA gene containing alkaline cleavage sites of the low pathogenic avian influenza virus strain are artificially synthesized, and BSPQI-HA-forward and BSPQI-HA-reverse as well as BSPQI-NA-forward and BSPQI-NA-reverse are used as upstream and downstream primers (containing BspQI enzyme digestion sites, as shown in Table 1) for PCR amplifications, respectively. The PCR amplification protocol is as follows: pre-degenerate at 94° C. for 5 minutes, enter the following cycle: degenerate at 94° C. for 45 seconds, anneal at 53° C. for 45 seconds and extend at 72° C. for 1 minute to 1 minute and 45 seconds, complete this cycle 30 times, and finally, extend at 72° C. for 10 minutes. At the end of reaction, the PCR product undergoes electrophoresis on 1.0% agarose gel.

4. Gel Cutting Recovery of the PCR Product

At the end of electrophoresis, the agarose gel of the target DNA fragment is cut off from gel under ultraviolet light, and DNA is recovered using a DNA rapid recovery kit. The specific method is as follows: cut off the target-DNA-containing agarose gel under a ultraviolet lamp, put the agarose gel in a sterile 1.5 ml EP tube, add Buffer DE-A (liquid gel) the volume of which is 3 times as much as that of the gel (100 mg=100 ul), mix uniformly and then heat at 75□, mix intermittently (for 2-3 minutes) until blocky gel is completely molten (about 6-8 minutes). Add Buffer DE-B (binding buffer) the volume of which is a half of that of the Buffer DE-A, and mix uniformly; add isopropanol the volume of which is equal to that of the gel when the recovered DNA fragment is less than 400 bp. Transfer the mixed solution to a DNA preparation tube, centrifuge for 1 minute at a rate of 12000×g, pour the waste solution in the collection tube. Place the preparation tube back into the collection tube, add 500 ul Buffer W1 (cleaning solution), centrifuge for 30 seconds at a rate of 12000×g, pour the waste solution in the collection tube. Place the preparation tube back into the collection tube, add 700 ul Buffer W2 (desalting solution), centrifuge for 1 minute at a rate of 12000×g, pour the waste solution in the collection tube, and wash the collection tube once again in the same way. Place the preparation tube back into the collection tube, and centrifuge in vacuum for 1 minute at a rate of 12000×g. Finally, place the preparation tube in the clean 1.5 ml EP tube, add 30 ul of de-ionized water to the center of a preparation membrane, stand at room temperature for 1 minute, centrifuge for 1 minute at a rate of 12000×g to elute DNA, and preserve the product at minus 20° C. for future use.

5. Enzyme Digestion, Ligation and Transformation

The abovementioned purified PCR product and a PBD vector (Zejun Li, et al. JVI, 2005, 79(18):12058-12064) are digested using BSPQI restriction endonuclease, the enzyme-digested product of the target fragments and the PBD plasmids is recovered using a gel extraction kit, and then, the PCR product after enzyme digestion is ligated to the enzyme-digested PBD vector using T4 ligase. The ligation product is transformed to a competent cell JM109 (Shanghai Solarbio Bioscience & Technology Co., LTD.), and the competent cell is then coated on an Amp-containing LB solid culture medium under aseptic conditions, followed by culture for 8-20 hours at 37° C.

6. Identification of the Recombinant Plasmids

A single colony on the LB solid culture medium is picked out and then placed in the test tube of an Amp-containing LB liquid culture medium that has a volume of about 3 ml, followed by shaking culture at 37° C. for 10 hours. The plasmids, which are extracted from the bacterial solution using an alkaline extraction method, are identified using a PCR method. The plasmids identified as being positive undergo sequencing, and also undergo sequence analysis using DNAstar sequence analysis software to determine the correctness of the sequences. The sequences respectively are nucleotide sequences as shown in SEQ ID NO.20-23, or amino acid sequences as shown in SEQ ID NO.24-27. In addition, the sequences of the various subtype HA and NA genes are also correct according to identification.

TABLE 1
Site-Directed Mutation Primers of NS2 and NP Genes and Universal
Primers of Influenza A Virus

| | Use | Serial No. | SEQ ID NO: |
|---|---|---|---|
| PR8-NS2-193F | Amino acid at the | TGGCGGTCACAATTAGGTCA | 1 |
| PR8-NS2-204R | 67^{th} site on NS2 is | TTGTGACCGCCATTTCTCGTTTCT | 2 |
| | mutated from E to S | | |
| PR8-NS2-215F | Amino acid at the | AGTTTTCAGAAATAAGATGGTT | 3 |
| PR8-NS2-224R | 74^{th} site on NS2 is | TCTGAAAACTTCTGACCTAATT | 4 |
| | mutated from E to S | | |
| PR8-NP-387F | Amino acid at the | AACGGCTGCACTGACTCACATGAT | 5 |
| PR8-NP-400R | 132^{nd} site on NP is | TCAGTGCAGCCGTTGCATCGTCACCA | 6 |
| | mutated from G to A | | |
| BSPQI-NP-forward | Universal primer of | CACACAGCTCTTCGGCCAGCAAAAGCAGGGTA | 7 |
| BSPQI-NP-reverse | NP gene of influenza | CACACAGCTCTTCTATTAGTAGAAACAAGGGTA | 8 |
| | A virus | TTTTT | |
| BSPQI-NS-forward | Universal primer of | CACACAGCTCTTCTATTAGCAAAAGCAGGGTG | 9 |
| BSPQI-NS-reverse | NS gene of influenza | CACACAGCTCTTCGGCCAGTAGAAACAAGGGT | 10 |
| | A virus | GTTTT | |
| BSPQI-NA-forward | Universal primer of | CACACAGCTCTTCTATTAGCAAAAGCAGGAGT | 11 |
| BSPQI-NA-reverse | NA gene of influenza | CACACAGCTCTTCGGCCAGTAGAAACAAGGAG | 12 |
| | A virus | TTTTTT | |
| BSPQI-HA-forward | Universal primer of | CACACAGCTCTTCTATTAGCAAAAGCAGGGG | 13 |
| BSPQI-HA-reverse | HA gene of influenza | CACACAGCTCTTCGGCCAGTAGAAACAAGGGT | 14 |
| | A virus | GTTTT | |
| H5-reverse | Mutation of HA | TAGTCCTCTTCTCTCTCCTTG | 15 |
| H5-forward | cleavage sites of | CAAGGAGAGAGAAGAGGACTA | 16 |
| | H5N1 highly | | |
| | pathogenic virus | | |
| H7-forward | Mutation of HA | CGAAATCCCAGGCCTATTTGGT | 17 |
| H7-reverse | cleavage sites of | ACCAAATAGGCCTGGGATTTCG | 18 |
| | H7N7 highly | | |
| | pathogenic virus | | |
| 12 bp reverse | Virus cDNA synthesis | AGCAAAAGCAGG | 19 |
| transcription | | | |
| primer | | | |

### Example 2

### Rescue of the Recombinant PR8 Mutant Virus

1. Preparation for Plasmid Transfection

The recombinant plasmids that are constructed using the aforementioned method are extracted using an ultra-pure plasmid extraction kit (OMEGA), including: PBD-(H1)HA, PBD-(H1)NA; PBD-(H3)HA, PBD-(H3)NA; PBD-(H4)HA, PBD-(H4)NA; PBD-(H5)HA, PBD-(H5)NA; PBD-(H6)HA, PBD-(H6)NA; PBD-(H7)HA, PBD-(H7)NA; PBD-(H9)HA, PBD-(H9)NA; PBD-(H10)HA, PBD-(H10)NA; PBD-PR8NS-NS2E67174S, PBD-PR8NS-NS2E67S, PBD-PR8NS-NS2E74S, PBD-PR8NP-G132A, PBD-PR8PB1, PBD-PR8PB2, PBD-PR8PA, PBD-PR8NP, PBD-PREM and PBD PR8NS, and the concentration of these plasmids is measured.

2. Acquisition of the Recombinant PR8 Mutant Virus by Rescue

The aforementioned plasmids are co-transfected to a 293T cell through liposome 2000 according to the designed combinations. 6 hours after transfection, the cell supernatant is discarded, 2 ml of OPTI-MEM is added, and the cell is put in a CO₂ incubator at 37° C. for culture for 72 hours. The transfected cell supernatant is treated with TPCK-Trypsin and then inoculated into 9-day to 11-day SPF chicken embryos (BEIJING MERIAL VITAL LABORATORY ANIMAL TECHNOLOGY CO., LTD.), the chicken embryos are sealed by paraffin and then put in an incubator at 37° C. for continuous incubation. 48 to 72 hours later, the chicken embryos are put under a temperature of 4° C. overnight, and then taken out to acquire chicken embryo allantoic fluids. The presence of hemagglutinin activity in the allantoic fluids is determined by a hemagglutination test.

The PR8 recombinant viruses and PR8 mutant recombinant viruses containing the HA and NA genes of the H1 subtype influenza virus are acquired by rescue in the present invention: H1N1-PR8 (1-PR8 for short), H1N1-PR8-NS2E67S(1-67 for short), H1N1-PR8-NS2E74S(1-74 for short), H1N1-PR8-NS2E67S/E74S(1-67/74 for short), H1N1-PR8-NP-G132A(1-132 for short) and H1N1-PR8-NPG132A-NS2E67S/E74S(1-132/67/74 for short).

The PR8 recombinant viruses and PR8 mutant recombinant viruses containing the HA and NA genes of the H3 subtype influenza virus are acquired by rescue in the present invention: H3N2-PR8 (3-PR8 for short), H3N2-PR8-NS2E67S(3-67 for short), H3N2-PR8-NS2E74S(3-74 for short), H3N2-PR8-NS2E67S/E74S(3-67174 for short), H3N2-PR8-NP-G132A(3-132 for short) and H3N2-PR8-NPG132A-NS2E67S/E74S(3-132/67174 for short).

The PR8 recombinant viruses and PR8 mutant recombinant viruses containing the HA and NA genes of the H4 subtype influenza virus are acquired by rescue in the present invention: H4N2-PR8(4-PR8 for short), H4N2-PR8-NS2E67S(4-67 for short), H4N2-PR8-NS2E74S(4-74 for short), H4N2-PR8-NS2E67S/E74S(4-67/74 for short), H4N2-PR8-NPG132A(4-132 for short) and H4N2-PR8-NPG132A-NS2E67S/E74S(4-132/67/74 for short).

The PR8 recombinant viruses and PR8 mutant recombinant viruses containing the HA and NA genes of the H5 subtype influenza virus are acquired by rescue in the present invention: H5N1-PR8(5-PR8 for short), H5N1-PR8-NS2E67S(5-67 for short), H5N1-PR8-NS2E74S(5-74 for short), H5N1-PR8-NS2E67S/E74S(5-67/74 for short), H5N1-PR8-NPG132A(5-132 for short) and H5N1-PR8-NPG132A-NS2E67S/E74S(5-132/67/74 for short).

The PR8 recombinant viruses and PR8 mutant recombinant viruses containing the HA and NA genes of the H6 subtype influenza virus are acquired by rescue in the present invention: H6N4-PR8(6-PR8 for short), H6N4-PR8-NS2E67S(6-67 for short), H6N4-PR8-NS2E74S(6-74 for short), H6N4-PR8-NS2E67S/E74S(6-67/74 for short), H6N4-PR8-NPG132A(6-132 for short) and H6N4-PR8-NPG132A-NS2E67S/E74S(6-132/67/74 for short).

The PR8 recombinant viruses and PR8 mutant recombinant viruses containing the HA and NA genes of the H7 subtype influenza virus are acquired by rescue in the present invention: H7N7-PR8(7-PR8 for short), H7N7-PR8-NS2E67S(7-67 for short), H7N7-PR8-NS2E74S(7-74 for short), H7N7-PR8-NS2E67S/E74S(7-67/74 for short), H7N7-PR8-NPG132A(7-132 for short) and H7N7-PR8-NPG132A-NS2E67S/E74S(7-132/67/74 for short).

The PR8 recombinant viruses and PR8 mutant recombinant viruses containing the HA and NA genes of the H9 subtype influenza virus are acquired by rescue in the present invention: H9N2-PR8(9-PR8 for short), H9N2-PR8-NS2E67S(9-67 for short), H9N2-PR8-NS2E74S(9-74 for short), H9N2-PR8-NS2E67S/E74S(9-67/74 for short), H9N2-PR8-NP132A(9-132 for short) and H9N2-PR8-NPG132A-NS2E67S/E74S(9-132167/74 for short).

The PR8 recombinant viruses and PR8 mutant recombinant viruses containing the HA and NA genes of the H10 subtype influenza virus are acquired by rescue in the present invention: H10N8-PR8(10-PR8 for short), H10N8-PR8-NS2E67S(10-67 for short), H10N8-PR8-NS2E74S (10-74 for short), H10N8-PR8-NS2E67S/E74S(10-67/74 for short), H10N8-PR8NP-G132A (10-132 for short) and H10N8-PR8-NPG132A-NS2E67S/E74S(10-132/67/74 for short).

3. Identification of the Recombinant Viruses

Total RNA of the allantoic fluids of the recombinant viruses is extracted using Trizol, and then undergoes reverse transcription using a 12-bp primer to acquire first-strand cDNA. By taking the first-strand cDNA as the template, BSPQI-HA-forward and BSPQI-HA-reverse, BSPQI-NA-forward and BSPQI-NA-reverse, BSPQI-NP-forward and BSPQI-NP-reverse, BSPQI-NS-forward and BSPQI-NS-reverse are used as upstream and downstream primers to amplify HA, NA, NP and NS fragments using a PCR method respectively, and these PCR products undergo sequencing after being purified. The sequencing results have confirmed that all the fragments contained in the PR8 mutant recombinant viruses are expected, and no unexpected mutation is found.

### Example 3

### Identification of the Growth Characteristics of the Rescued Recombinant Viruses

1. Determination of EID₅₀ of the Rescued Recombinant Viruses

The virus-containing chicken embryo allantoic fluids undergo 10-fold dilutions, and the chicken embryo allantoic fluids that are diluted based on the dilutabilities from 10⁻⁵ to 10⁻⁹ are respectively inoculated to three 9-day to 11-day SPF chicken embryos for continuous incubation at 37° C. for 48 hours. Whether the chicken embryo allantoic fluids are infected is judged by determining the hemagglutinin activities of the infected embryo allantoic fluids, and EID₅₀ (median embryo infective dose) is calculated using a Reed-Muench method. The determination results of EID₅₀ of the recombinant viruses are shown in Table 2 (wherein the virus diluents have a volume of 100 ul).

TABLE 2
EID₅₀ of the Recombinant Virus Strains

| Name of the | | | | | | | | | |
|---|---|---|---|---|---|---|---|---|---|
| Recombinant | | HA, NA Donor Viruses | | | | | | | |
| Viruses | Internal Gene Donor Viruses | H1N1 | H3N2 | H4N6 | H5N2 | H6N4 | H7N7 | H9N2 | H10N8 |
| x-67 | PR8-NS2E67S | 10^{7.3} | 10^{7.0} | 10^{7.3} | 10^{7.0} | 10^{7.0} | 10^{7.8} | 10^{7.0} | 10^{7.5} |
| x-74 | PR8-NS2E74S | 10^{7.3} | 10^{7.5} | 10^{7.3} | 10^{7.3} | 10^{7.5} | 10^{7.0} | 10^{7.8} | 10^{7.3} |
| x-67/74 | PR8-NS2E67/74S | 10^{7.5} | 10^{7.8} | 10^{7.0} | 107^{8.0} | 10^{7.8} | 10^{7.5} | 10^{7.8} | 10^{7.8} |
| x-132 | PR8-NPG132A | 10^{7.8} | 10^{7.5} | 10^{7.3} | 10^{7.3} | 10^{7.0} | 10^{7.0} | 10^{7.0} | 10^{7.5} |
| x-132/67/74 | PR8-NPG132A-NS2E67S/E74S | 10^{7.5} | 10^{7.5} | 10^{7.8} | 10^{7.3} | 10^{7.3} | 10^{7.0} | 10^{7.5} | 10^{7.8} |
| x-PR8 | PR8 | 10^{7.3} | 10^{7.8} | 10^{7.3} | 10^{7.0} | 10^{7.0} | 10^{7.0} | 10^{7.3} | 10^{7.3} |

2. Determination of TCID₅₀ of the Rescued Recombinant Viruses

10-fold dilutions start from 1: 10⁻², the recombinant viruses having different dilutabilities are inoculated to a 48-well plate on which monolayer MDCK cells grow, and the inoculation procedure is as follows: at first, clean the MDCK cells twice with PBS, then add 100 ul of virus to each well, repeat this operation 3 times for every dilutability, put the 48-well plate in a CO₂ incubator at 37° C. for the purpose that viruses are adsorbed onto the cells, shake the culture plate once at an interval of 20 minutes, discard the liquid in the cell culture plate 1.5-2.5 hours later, wash the cells twice with PBS, and then add 300 ul of serum-free medium containing TPCK-Trypsin, continuously culture the cells in the CO₂ incubator for 72 hours, determine the hemagglutinin activity in every well, and calculate TCID₅₀ (median tissue culture infective dose) using a Reed-Muench method. The determination results of TCID₅₀ of the recombinant viruses are shown in Table 3 (wherein, the virus diluents have a volume of 100 ul).

TABLE 3
TCID₅₀ of the Recombinant Virus Strains

| Name of the | | HA and NA Donor Viruses | | | | | | | |
|---|---|---|---|---|---|---|---|---|---|
| Recombinant Viruses | Internal Gene Donor Viruses | H1N1 | H3N2 | H4N6 | H5N2 | H6N4 | H7N7 | H9N2 | H10N8 |
| x-67 | PR8-NS2E67S | 10^{6.0} | 10^{5.5} | 10^{5.3} | 10^{6.0} | 10^{5.5} | 10^{5.3} | 10^{5.5} | 10^{5.0} |
| x-74 | PR8-NS2E74S | 10^{5.8} | 10^{5.0} | 10^{5.3} | 10^{5.8} | 10^{5.5} | 10^{5.8} | 10^{5.5} | 10^{5.5} |
| x-67/74 | PR8-NS2E67/74S | 10^{5.3} | 10^{5.3} | 10^{4.5} | 10^{5.0} | 10^{5.3} | 10^{5.3} | 10^{5.0} | 10^{5.3} |
| x-132 | PR8-NPG132A | 10^{5.8} | 10^{5.5} | 10^{5.3} | 10^{5.8} | 10^{5.8} | 10^{5.5} | 10^{5.3} | 10^{5.8} |
| x-132/67/74 | PR8-NPG132A-NS2E67S/E74S | 10^{5.3} | 10^{5.5} | 10^{5.0} | 10^{5.3} | 10^{5.3} | 10^{5.0} | 10^{5.5} | 10^{5.8} |
| x-PR8 | PR8 | 10^{5.8} | 10^{5.5} | 10^{5.3} | 10^{5.5} | 10^{5.5} | 10^{5.0} | 10^{5.0} | 10^{5.3} |

3. Growth Characteristic Comparison of the Rescued Recombinant Viruses on Chicken Embryos

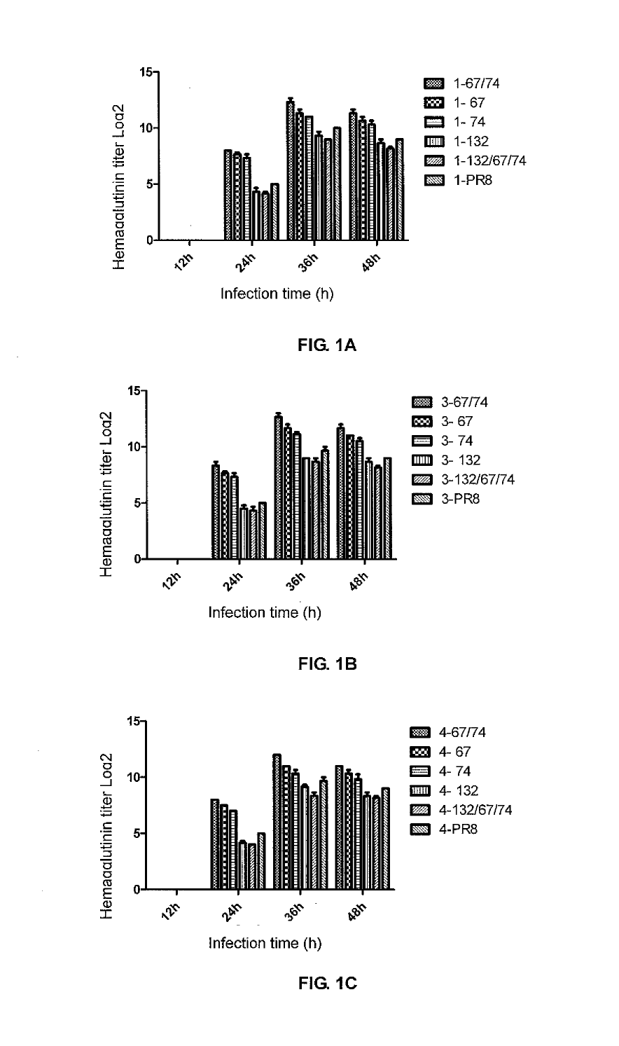

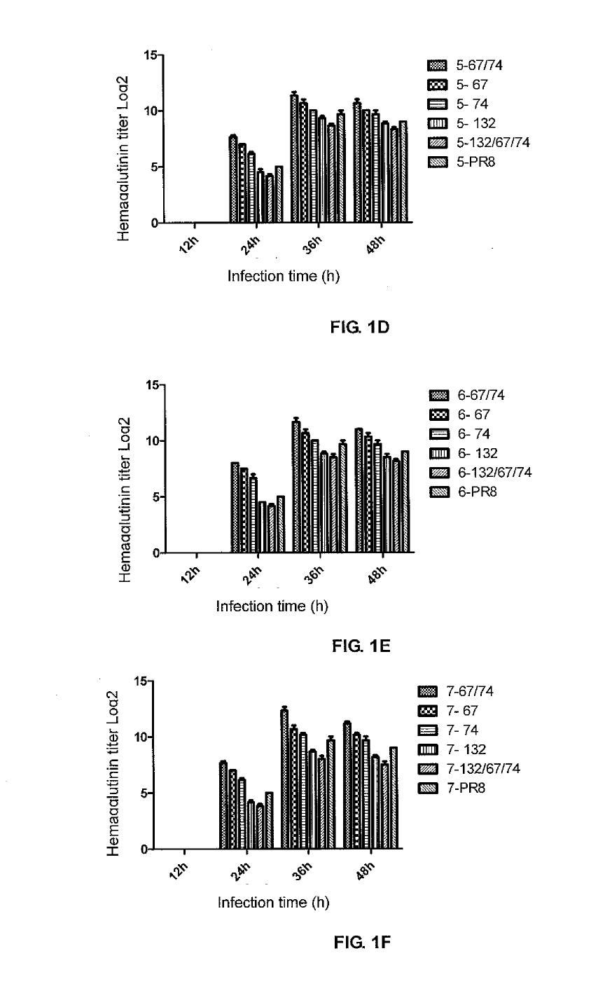

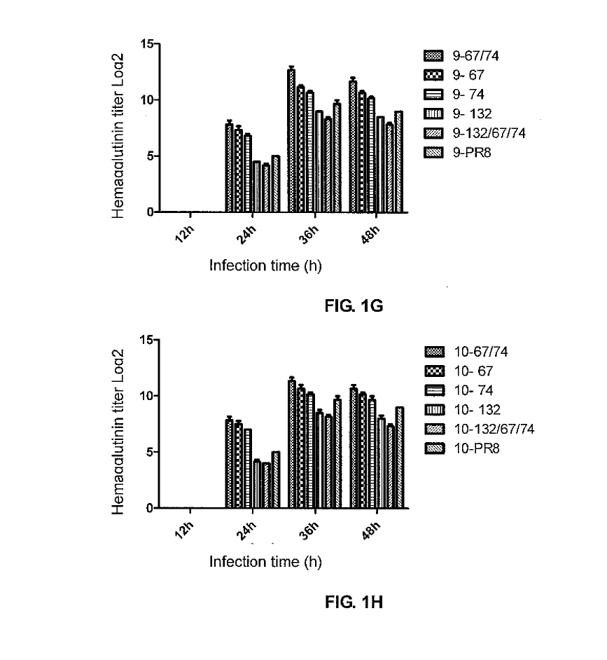

100 ul of recombinant virus diluents with 100EID₅₀ are inoculated into 9-day to 11-day SPF chicken embryos, and 6 hours, 12 hours, 24 hours, 36 hours and 48 hours after inoculation, 3 SPF chicken embryos are taken out respectively, their allantoic fluids are collected and the hemagglutinin titers thereof are determined. The hemagglutinin titers of different subtype recombinant viruses after multiplication on the chicken embryos present similar results, the allantoic fluids of the virus-inoculated chicken embryos all have no hemagglutinin activity within 12 hours after virus inoculation, the recombinant virus (HxNy-PR8-NS2-E67S/E74S, x-67/74 for short) containing six internal genes of the mutant virus PR8-NS2-E67/743 has the highest hemagglutinin titer within 24-48 hours after virus inoculation, and the other recombinant viruses with their hemagglutinin titers in a descending order are as follows: the recombinant virus (HxNy-PR8-NS2-E67S, x-67 for short) containing six internal genes of the mutant virus PR8-NS2-E67S, the recombinant virus (HxNy-PR8-NS2-E67S, x-74 for short) containing six internal genes of the PR8-NS2-E74S, the recombinant virus (HxNy-PR8, x-PR8 for short) containing six internal genes of the PR8 virus, the recombinant virus (HxNy-PR8-NP-G132A, x-132 for short) containing six internal genes of PR8-NP-G132A, and the recombinant virus (HxNy-PR8-NP-G132A-NS2-E67S/E74S, x-132/67/74 for short) containing six internal genes of PR8-NP-G132A-NS2-E67S/E74S. As shown in FIG. 1, the aforementioned results are intuitively shown by H1N1, H3N2, H4N2, H5N1, H6N4, H7N7, H9N2 and H10N8 subtype recombinant viruses.

4. Growth Characteristic Comparison of the Rescued Recombinant Viruses and PR8 on MDCK Cells

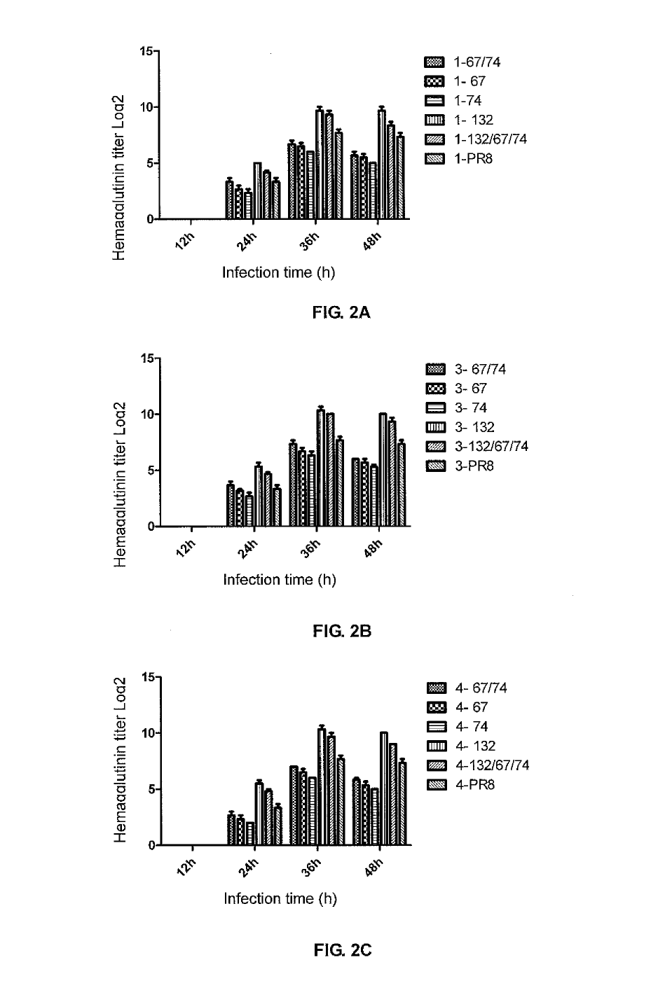

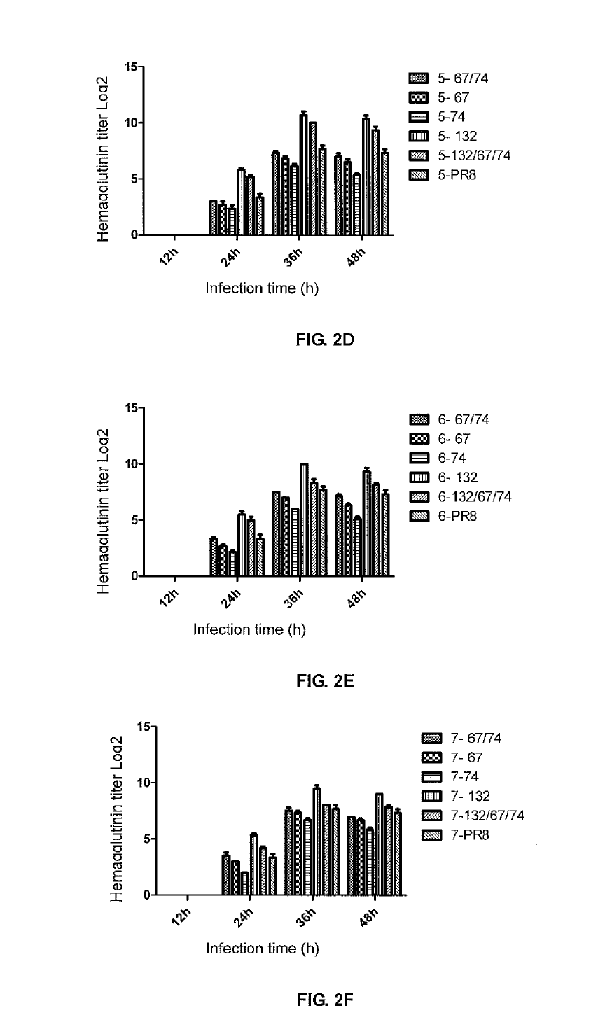

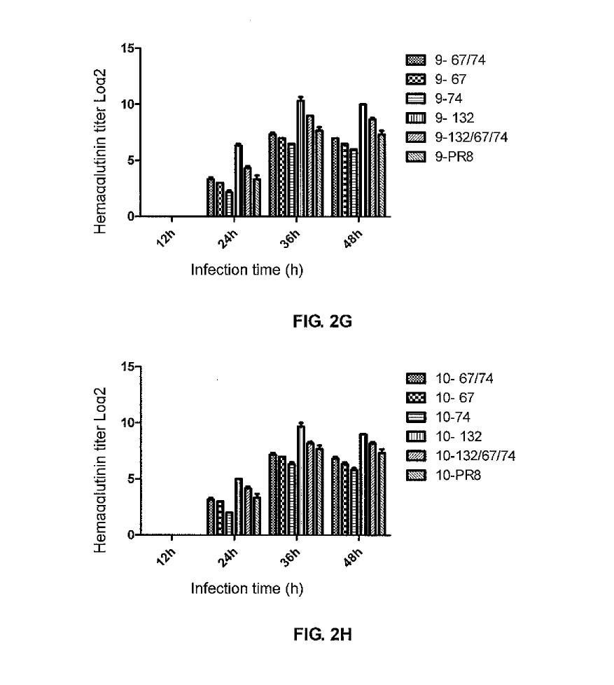

200 ul of recombinant virus diluents with 100TCID₅₀ are inoculated into MDCK cells in a T25 cell culture flask. 6 hours, 12 hours, 24 hours, 36 hours and 48 hours after inoculation, the cell supernatants are collected respectively and the hemagglutination titers thereof are determined, and the growth conditions of the recombinant viruses on the MDCK cells are compared. The hemagglutinin titers of different subtype recombinant viruses after multiplication on the cells present similar results, the recombinant virus-infected cell supernatants all have no hemagglutinin activity within 12 hours after virus inoculation. 24 hours, 36 hours and 48 hours after virus inoculation, the recombinant virus (x-132) containing six internal genes of PR8-NP-G132A has the highest hemagglutinin titer, and the other recombinant viruses with their hemagglutinin titers in a descending order are as follows: the recombinant virus (x-132167174) containing six internal genes of PR8-NP-G132A-NS2-E67S/E74S, the recombinant virus (x-PR8) containing six internal genes of the PR8 virus, the recombinant virus (x-67/74) containing six internal genes of the mutant virus PR8-NS2-E67/74S, the recombinant virus (x-67) containing six internal genes of the mutant virus PR8-NS2-E67S, and the recombinant virus (x-74) containing six internal genes of PR8-NS2-E74S. As shown in FIG. 2, the aforementioned results are intuitively shown by H1N1, H3N2, H4N2, H5N1, H6N4, H7N7, H9N2 and H10N8 subtype recombinant viruses.

The embodiments described above are merely for illustrating the ways of practicing the present invention in a specific and detailed manner, but could not be understood as limiting the patent scope of the present invention therefrom. It shall be noted that, many modifications and improvements could also be made by those ordinary skilled in this art without departing from the concept of the present invention, and these modifications and improvements shall fall within the scope of protection of the present invention. Accordingly, the patent scope of the present invention shall be subject to the claims appended.

1. A PR8 recombinant influenza virus comprising
an HA and/or NA gene of an influenza virus, and
6 internal genes (PB1, PB2, PA, NP, M and NS genes) of PR8 virus,
wherein the NS and/or NP gene comprises the following mutation sites:
the NS2 protein encoded by the NS gene has an E67S point mutation, E74S point mutation or E67S/E74S point mutation, and
the NP protein encoded by the NP gene has a G132A point mutation, and
wherein the influenza virus is one selected from the group consisting of an H1 subtype influenza virus other than PR8 virus, an H3 subtype influenza virus, an H4 subtype influenza virus, an H5 subtype influenza virus, an 116 subtype influenza virus, an H7 subtype influenza virus, an H9 subtype influenza virus, and an H10 subtype influenza virus.

2. The PR8 recombinant influenza virus of claim 1, wherein the influenza virus is H3 subtype influenza virus.

3. The PR8 recombinant influenza virus of claim 1, wherein the influenza virus is H4 subtype influenza virus.

4. The PR8 recombinant influenza virus of claim 1, wherein the influenza virus is H5 subtype influenza virus.

5. The PR8 recombinant influenza virus of claim 1, wherein the influenza virus is H6 subtype influenza virus.

6. The PR8 recombinant influenza virus of claim 1, wherein the influenza virus is subtype influenza virus.

7. The PR8 recombinant influenza virus of claim 1, wherein the influenza virus is subtype influenza virus.

8. The PR8 recombinant influenza virus of claim 1, wherein the influenza virus is H10 subtype influenza virus.

9. A method for preparing a PR8 recombinant influenza virus, comprising:
constructing a recombinant plasmid comprising the HA and NA genes of the H1 other than PR8 virus, H3, H4, H5, H6, H7, H9 or H10 subtype influenza virus;
constructing a recombinant plasmid comprising a PR8 virus mutant gene fragment, the PR8 virus mutant gene fragment being selected from the group consisting of the following mutant NS or NP gene fragments: a PR8 virus NS gene fragment encoding the NS2 protein containing the E67S point mutation, E74S point mutation or E67S/E74S point mutation, and a PR8 virus NP gene fragment encoding the NP protein containing the G132A point mutation;
co-transfecting the recombinant plasmid of the HA and NA genes, the recombinant plasmid comprising the PR8 virus mutant gene fragment, and the plasmids respectively comprising the PA, PB1, PB2, M, NP or NS internal gene of PR8 virus, into a 293T cell, and culturing the transfected cell;
inoculating the cultured cell supernatant into chicken embryos, and culturing the chicken embryos in an incubator for a proper time period to acquire chicken embryo allantoic fluid, detecting the hemagglutination condition of the allantoic fluid, and in case of the presence of hemagglutinin activity, determining the absence of unexpected mutations by sequence analysis to acquire the PR8 recombinant influenza virus.

10. A method for immunizing a subject against an influenza viral infection comprising: administering to the subject an effective amount of a vaccine prepared by using the PR8 recombinant influenza virus according to claim 1.

11. The PR8 recombinant influenza virus of claim 1, wherein the influenza virus is H1 subtype influenza virus other than PR8 virus.

12. The method for preparing the PR8 recombinant influenza virus according to claim 9, wherein the plasmid comprising the HA and NA genes of H1 subtype influenza virus other than PR8 virus.

13. The method for preparing the PR8 recombinant influenza virus according to claim 9, wherein the plasmid comprising the HA and NA genes of H3 subtype influenza virus.

14. The method for preparing the PR8 recombinant influenza virus according to claim 9, wherein the plasmid comprising the HA and NA genes of H4 subtype influenza virus.

15. The method for preparing the PR8 recombinant influenza virus according to claim 9, wherein the plasmid comprising the HA and NA genes of H5 subtype influenza virus.

16. The method for preparing the PR8 recombinant influenza virus according to claim 9, wherein the plasmid comprising the HA and NA genes of H6 subtype influenza virus.

17. The method for preparing the PR8 recombinant influenza virus according to claim 9, wherein the plasmid comprising the HA and NA genes of H7 subtype influenza virus.

18. The method for preparing the PR8 recombinant influenza virus according to claim 9, wherein the plasmid comprising the HA and NA genes of H9 subtype influenza virus.

19. The method for preparing the PR8 recombinant influenza virus according to claim 9, wherein the plasmid comprising the HA and NA genes of H10 subtype influenza virus.
